(19) United States
(12) Patent Application Publication
(10) Pub. No.: US 2013/0338936 A1
(43) Pub. Date: Dec. 19, 2013

(54) METHOD AND SYSTEM FOR CARBON ACCOUNTING

(71) Applicant: National Taiwan Normal University, Taipei City (TW)

(72) Inventors: Jon-Chao HONG, Taipei City (TW); Hungy HONG, Taipei City (TW)

(21) Appl. No.: 13/910,271

(22) Filed: Jun. 5, 2013

(30) Foreign Application Priority Data
Jun. 15, 2012 (TW) 101121551

(51) Int. Cl. G01N 33/00

(52) U.S. Cl. CPC G01N 33/0027
USPC 702/24

(57) ABSTRACT

In a method for carbon accounting, an interface module of a server is configured to receive carbon emission data of a target, the carbon emission data including at least one carbon footprint activity of the target and an estimated amount associated with the carbon footprint activity. A calculating module of the server is configured to calculate carbon emission associated with the target. An alert module of the server is configured to generate a high emission alert when the carbon emission calculated by the calculating module is higher than a first predetermined value, and to generate a low emission alert when the carbon emission is lower than a second predetermined value.

### CROSS-REFERENCE TO RELATED APPLICATION

This application claims priority of Taiwanese Application No. 101121551, filed on Jun. 15, 2012.

### BACKGROUND OF THE INVENTION

1. Field of the Invention

The invention relates to a method and a system for performing carbon accounting for a target that may include multiple individuals.

2. Description of the Related Art

The term “carbon footprint” generally refers to “a measure of the total amount of carbon dioxide (CO₂) and methane (CH₄) emissions, calculated as carbon dioxide equivalent (CO₂e)”. Since carbon dioxide is a primary greenhouse gas, carbon emissions a main contributor global warming phenomenon.

Carbon accounting refers to procedures for measuring carbon emission of an entity. For example, an electric utility may provide customers with information regarding carbon emission associated with electricity consumption of the customers.

However, a more detailed scheme that involves measuring carbon emissions caused by daily activities (usually referred to as carbon footprint activities), rather than solely relying on electricity usage, may be more preferable. Moreover, in addition to measuring carbon emissions, it would be beneficial to have a system that also performs carbon emission analysis to help users thereof gain awareness about global warming, and to alert a user when his/her carbon footprint activities have caused excessive carbon emissions.

### SUMMARY OF THE INVENTION

Therefore, the object of the present invention is to provide a method capable of providing a more detailed carbon accounting procedure and carbon emission analysis for a target.

Accordingly, a method for carbon accounting of the present invention is to be implemented using a server. The method comprises:

configuring an interface module of the server to receive carbon emission data of a target, the carbon emission data including at least one carbon footprint activity of the target and an estimated amount associated with the carbon footprint activity;

configuring a calculating module of the server to calculate carbon emission that is associated with the target based on the carbon emission data of the target;

configuring an alert module of the server to generate a high emission alert when the carbon emission calculated by the calculating module is higher than a first predetermined value; and

configuring the alert module of the server to generate a low emission alert when the carbon emission calculated by the calculating module is lower than a second predetermined value.

Another object of the present invention is to provide a system that is capable of implementing the abovementioned method.

Accordingly, a system for carbon accounting of the present invention comprises an interface module, a calculating module and an alert module.

The interface module receives carbon emission data of a target. Preferably, the carbon emission data includes at least one carbon footprint activity of the target and an estimated amount associated with the carbon footprint activity.

The calculating module is coupled to the interface module and calculates carbon emission that is associated with the target based on the carbon emission data of the target.

The alert module is coupled to the calculating module, and is configured to generate a high emission alert when the carbon emission calculated by the calculating module is higher than a first predetermined value, and a low emission alert when the carbon emission calculated by the calculating module is lower than a second predetermined value.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic diagram of the preferred embodiment of a system for carbon accounting according to the invention;

FIG. 2 is a schematic diagram of an interface that is generated by the system for interacting with a target; and

FIG. 3 is a flowchart illustrating the preferred embodiment of a method for carbon accounting according to the invention.

### DETAILED DESCRIPTION OF THE PREFERRED EMBODIMENT

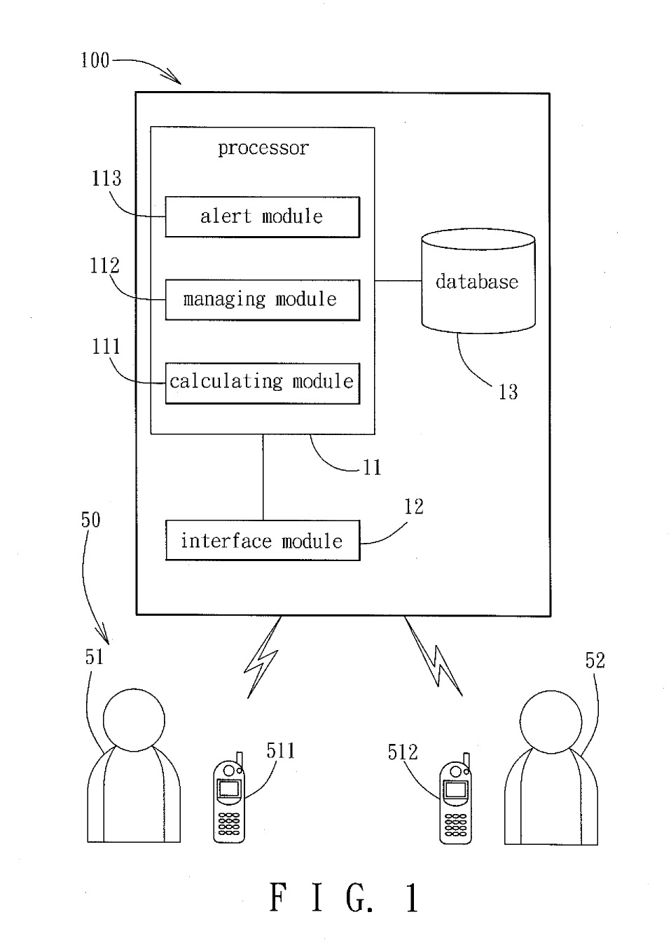

FIG. 1 illustrates the preferred embodiment of a system 100 for carbon accounting, according to the present invention. The system 100 is configured to interact with a target 50 that may include multiple individuals.

Each of the individuals (for example, in this embodiment two individuals 51 and 52 are present) has respective carbon emission data associated with his/her daily carbon footprint activities, and is able to communicate with the system 100 using a respective communication device (511, 512). The communication devices 511 and 512 maybe arbitrary electronic devices that are configured to establish a connection with the system 100, such as personal computers and mobile communication devices.

The system 100, which is a web server is this embodiment, comprises a processor 11, an interface module 12, and a database 13 that are coupled to one another.

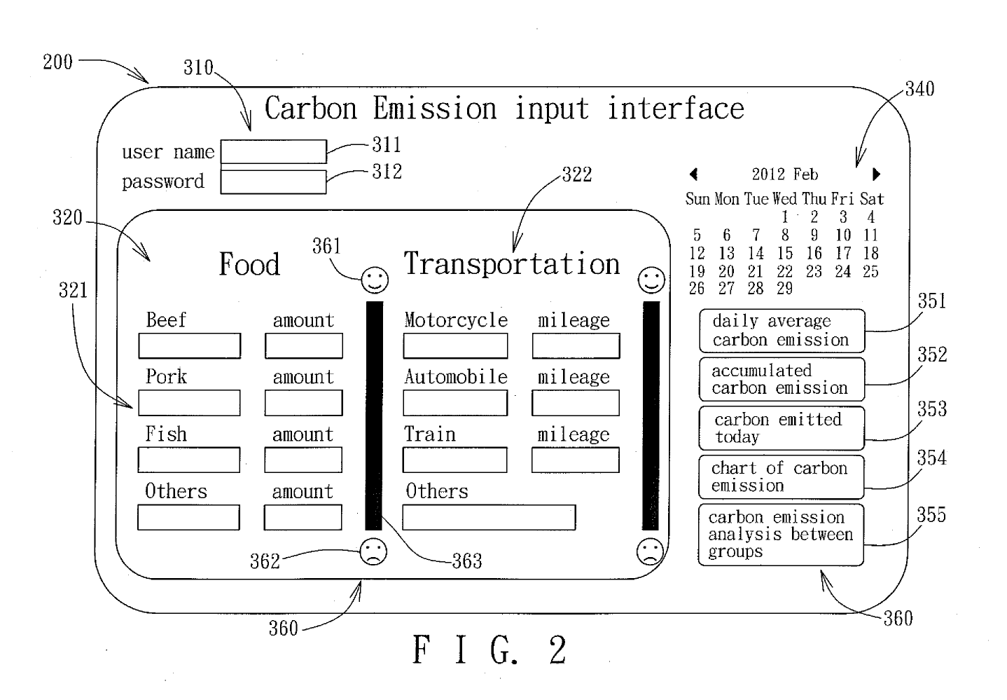

The processor 11 is configured to include a calculating module 111, a managing module 112 and an alert module 113. The interface module 12 is configured to generate a user interface 200 for interacting with the target 50. An exemplary interface 200, which may appear on displays of the communication devices 511 and 512, is illustrated in FIG. 2.

The database 13 stores account information of the target 50 and each of the individuals 51 and 52 therein. The account information may include an account name and password of each of the individuals, personal information such as contact information, carbon emission data associated with each of the individuals, etc. In some embodiments, the account information of each individual includes grouping information that indicates one of a plurality of groups (e.g., classes in a school) to which that individual belongs.

The target 50 may attempt to log into the system 100 by inputting account information thereof via a login area 310 having login fields 311 and 312. The inputted account information is compared with information in the database 13 for verification. The target 50 is successfully logged in when the verification of the account information is completed. While logged in, all and any information inputted by the target 50 can be associated with the account of the target 50 and stored in the database 13.

Preferably, even when the target 50 is not logged in the system 100, functions thereof will still be available to the target 50, but information may not be stored in the database 13.

In this embodiment, the interface module 12 receives the carbon emission data of the target 50 by allowing the target 50 to input the carbon emission data via the interface 200. The carbon emission data may include at least one carbon footprint activity of the target 50, and an estimated amount associated with that carbon footprint activity.

The carbon footprint activity may include, but is not limited to, at least one of food ingestion by the target 50, transportation taken by the target 50 and energy consumption associated with the target 50. The above carbon footprint activities are selected for being common activities in daily lives and for contributing a large portion of total carbon emission (according to statistics taken from the report “livestock's long shadow” released by Food and Agriculture Organization of the United Nations (FAO), livestock are responsible for 18% of greenhouse gas emissions, while transportation are responsible for 13.5%). Other related carbon footprint activity may also be included for providing a more detailed carbon accounting service.

The interface 200 further includes a carbon emission data input area 320. As shown in FIG. 2, fields 321 allow the target 50 to input a specific type of food, along with the amount of food that is ingested by the target 50. Table 1 lists some of the more common types of food, and the corresponding carbon emission that is attributed to producing a certain amount (e.g., one kilogram) of each type of food.

TABLE 1

| Food type | Carbon emission (Kg) per kilogram |
|---|---|
| Beef | 2.296 |
| Pork | 0.86 |
| Fish | 1.926 |
| Vegetable | 0.355 |

Similarly, fields 322 allow the target 50 to input type of transportation, along with the amount of time and/or mileage that is taken by the target 50. Table 2 lists some of the more common types of transportation means, and the corresponding carbon emission that is attributed to taking a certain mileage (e.g., one kilometer) of each type of transportation.

TABLE 2

| | | Carbon emission (Kg) |
|---|---|---|
| Transportation | Unit | per unit |
| Motorcycle | Kilometer | 0.0605 |
| Automobile | Kilometer | 0.236 |
| Train | Kilometer | 0.06 |
| Taiwan High Speed Rail | Kilometer | 0.05 |
| Mass Rapid Transit | Kilometer | 0.1631 |
| (MRT) | | |
| Bus | Kilometer | 0.0352 |

The interface 200 may further include fields that allow the target 50 to input type of energy consumption and/or electrical appliances used, along with the amount of time and/or usage associated with the target 50. Table 3 lists some of the more common types of energy consumption and electrical appliances, and the corresponding carbon emissions.

TABLE 3

| Energy consumption/ | | Carbon emission (Kg) |
|---|---|---|
| Electrical appliances | Unit | per unit |
| Tap water | Cubic meter | 0.19 |
| Natural gas | Cubic meter | 2.09 |
| Liquefied petroleum gas | Kilogram | 3.19 |
| Electric fan | Hour | 0.0455 |
| Air conditioner | Hour | 0.621 |
| Dehumidifier | Hour | 0.1967 |
| Air purifier | Hour | 0.0414 |

The interface 200 may further include a date-setting area 340 for allowing the target 50 to associate the inputted carbon emission data to a specific date.

Based on the carbon emission data inputted by the target 50, the calculating module 111 is configured to calculate the carbon emission that is associated with the target 50 for each of the carbon footprint activities. For example, consider an individual who conducted the following carbon footprint activities during a week, as listed in the following table:

TABLE 4

| Carbon | | | | |
|---|---|---|---|---|
| Footprint | | | Carbon emission | Resulted carbon |
| Activity | Unit | | (Kg) per unit | emission (Kg) |
| Beef | 1 | (Kg) | 2.296 | 2.296 |
| Pork | 0.5 | (Kg) | 0.86 | 0.43 |
| Fish | 0.1 | (Kg) | 1.926 | 0.1926 |
| Vegetable | 2.5 | (Kg) | 0.355 | 0.8875 |
| Motorcycle | 20 | (Km) | 0.0605 | 1.21 |
| Train | 20 | (Km) | 0.06 | 1.2 |
| MRT | 2 | (Km) | 0.1631 | 0.3262 |
| Tap water | 3 | (cubic meter) | 0.19 | 0.57 |
| Natural gas | 1.5 | (cubic meter) | 2.09 | 3.135 |
| Electric fan | 3 | (Hrs) | 0.0455 | 0.1365 |
| Air purifier | 3 | (Hrs) | 0.0414 | 0.1242 |
| | | | Total carbon | 10.508 (Kg) |
| | | | emission | |

The total carbon emission is then obtained by the calculating module 111 using the following equation:

$${CO}_{2 , total} = {\sum }_{i = 1}^{n} {CO}_{2 , per \_ i} \times {C}_{i}$$

Where CO_{2,total} a represents the total carbon emission, CO_{2,per}_{_{—}}ᵢ represents carbon emitted for each unit of the carbon footprint activity undertaken, and Cᵢ represents the amount of a specific carbon footprint activity.

Once the total carbon emission associated with the target 50 is obtained, the calculating module 111 is configured to compare the total carbon emission with a first predetermined value (e.g., a ceiling value) and a second predetermined value (e.g., a floor value). When the carbon emission calculated by the calculating module 111 is higher than the first predetermined value, the alert module 113 generate a high emission alert indicating that the carbon emission associated with the target 50 may be too high. Similarly, when the carbon emission calculated by the calculating module 111 is lower than the second predetermined value, the alert module 113 generate a low emission alert indicating that the carbon emission associated with the target 50 is favorable.

In this embodiment, the interface 200 may include an alert area 360 having a low emission alert indicator 361 (in the form of a green smiling face), a carbon emission gauge 362 which gives a graphical indication of the carbon emission and the first and second predetermined values, and a high emission alert indicator 363 (in the form of a red sad face).

The managing module 112 is configured to perform a number of management operations for managing the account of the target 50 based on the carbon emission data of the target 50. In some embodiments, the managing module 112 performs the management operations for managing accounts of each of the individuals 51 and 52 based on the respective carbon emission data of the individuals 51 and 52.

The management operation may include plotting a chart of carbon emission with respect to time, obtaining an average carbon emission over a period of time (e.g., one week), and obtaining an accumulated carbon emission over a period of time. The interface 200 may include a management area 350 that has a number of management buttons 351 to 355, each being associated with one particular management operation. For example, when button 351 is selected, the average carbon emission is calculated and displayed. Similarly, button 352 is for displaying the accumulated carbon emission, button 353 is for displaying the carbon emission associated with the current date, and button 354 is for displaying the chart of carbon emission. Button 355 is for enabling the managing module 112 to compare the accounts of the individuals 51 and 52 for carbon emission analysis.

In some embodiments, the carbon emission analysis involves ranking the individuals 51 and 52 according to the carbon emissions. In some embodiments, the carbon emission analysis involves ranking the groups further according to the grouping information. For example, when the groups are classes in a school, the button 355 may then be associated with determining which class has the most/least carbon emission within the school. Such carbon emission analysis involving ranking enables competition between individuals/groups, and rewards may be awarded to encourage efforts toward creating less carbon emission.

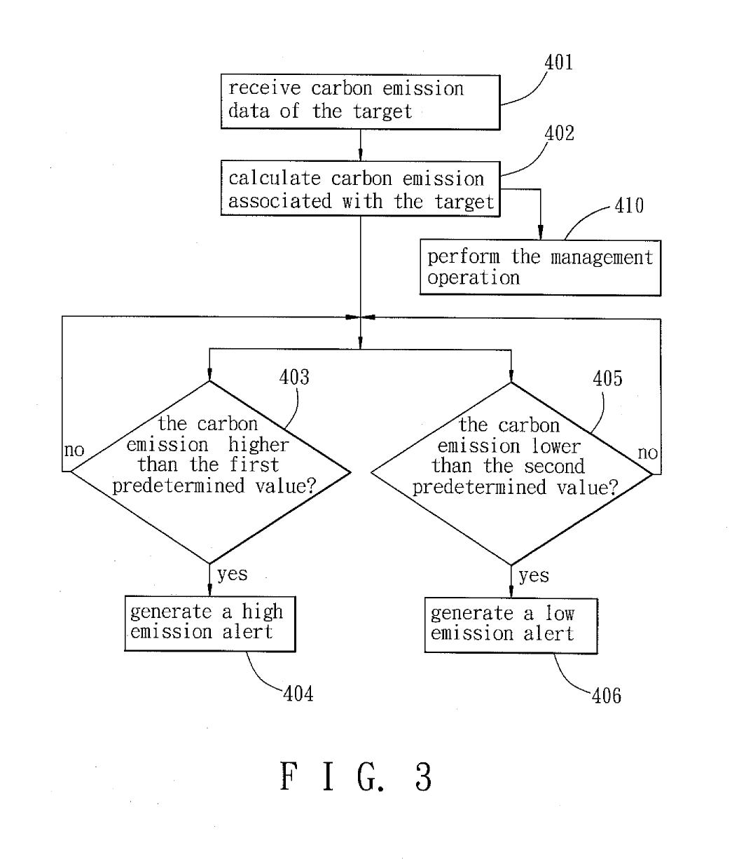

FIG. 3 illustrates a method for carbon accounting that is to be implemented using the system 100.

In step 401, the interface module 101 receives carbon emission data of the target. The calculating module 111 then calculates carbon emission associated with the target 50 in step 402, based on the carbon emission data of the target 50.

The alert module 113 compares the carbon emission calculated by the calculating module 111 with the predetermined values in steps 403 and 405, respectively, and generates the corresponding emission alert in steps 404 and 406. The managing module 112 performs the management operations in step 410.

To sum up, embodiments of this invention have a number of effects. For example, by inputting carbon emission data into the system 100, the target 50 is able to obtain the accumulated carbon emission that is associated with daily activities of the target 50. Thus, each individual of the target 50 may be able to have a more concrete understanding about carbon emission and subsequently awareness about global warming. Further, the system 100 is able to determine carbon emission levels attributed to the carbon footprint activities done by the target 50, and to generate corresponding alerts when the carbon emission level thus determined is deemed excessive or favorably low.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A method for carbon accounting to be implemented using a server and comprising:
configuring an interface module of the server to receive carbon emission data of a target, wherein the carbon emission data includes at least one carbon footprint activity of the target and an estimated amount associated with the carbon footprint activity;
configuring a calculating module of the server to calculate carbon emission that is associated with the target based on the carbon emission data of the target;
configuring an alert module of the server to generate a high emission alert when the carbon emission calculated by the calculating module is higher than a first predetermined value; and
configuring the alert module of the server to generate a low emission alert when the carbon emission calculated by the calculating module is lower than a second predetermined value.

2. The method of claim 1, further comprising configuring a managing module of the server to perform a management operation for managing an account of the target based on the carbon emission data of the target, wherein the management operation includes at least one of plotting a chart of carbon emission with respect to time, obtaining an average carbon emission over a period of time, and obtaining an accumulated carbon emission over a period of time.

3. The method of claim 1, the target including multiple individuals that have respective carbon emission data, said method further comprising:
configuring a managing module of the server to perform a management operation for managing an account of each of the individuals based on the respective carbon emission data of the individuals, wherein the management operation includes at least one of plotting a chart of carbon emission with respect to time, obtaining an average carbon emission over a period of time, and obtaining an accumulated carbon emission over a period of time.

4. The method of claim 3, further comprising configuring the managing module of the server to compare the accounts of the individuals for carbon emission analysis.

5. The method of claim 3, further comprising configuring the managing module of the server to rank the individuals according to the carbon emissions calculated by the calculating module.

6. The method of claim 1, wherein the carbon footprint activity includes at least one of food ingestion by the target, transportation taken by the target and energy consumption associated with the target.

7. The method of claim 1, wherein the server is a web server.

8. A system for carbon accounting, comprising:
an interface module that receives carbon emission data of a target, wherein the carbon emission data includes at least one carbon footprint activity of the target and an estimated amount associated with the carbon footprint activity;
a calculating module that is coupled to said interface module and that calculates carbon emission associated with the target, based on the carbon emission data of the target; and
an alert module that is coupled to said calculating module, and that is configured to generate a high emission alert when the carbon emission calculated by said calculating module is higher than a first predetermined value, and a low emission alert when the carbon emission calculated by said calculating module is lower than a second predetermined value.

9. The system of claim 8, further comprising a managing module that performs a management operation for managing an account of the target based on the carbon emission data of the target;
wherein the management operation includes at least one of plotting a chart of carbon emission with respect to time, obtaining an average carbon emission over a period of time, and obtaining an accumulated carbon emission over a period of time.

10. The system of claim 8, the target including multiple individuals that have respective carbon emission data, said system further comprising a managing module that performs a management operation for managing an account of each of the individuals based on the respective carbon emission data of the individuals;
wherein the management operation includes at least one of plotting a chart of carbon emission with respect to time, obtaining an average carbon emission over a period of time, and obtaining an accumulated carbon emission over a period of time.

11. The system of claim 10, wherein said managing module further compares the accounts of the individuals for carbon emission analysis.

12. The system of claim 10, wherein said managing module further ranks the individuals according to the carbon emissions calculated by said calculating module.

13. The system of claim 8, wherein the carbon footprint activity includes at least one of food ingestion by the target, transportation taken by the target and energy consumption associated with the target.

14. The system of claim 8, which is a web server.
